SEQUENCE LISTING (6 sequences)

SEQ ID NO: 1 - DNA, 20 bp, synthetic construct
  source 1..20: mol_type = other DNA
agagtttgatcctggctcag

SEQ ID NO: 2 - DNA, 19 bp, synthetic construct
  source 1..19: mol_type = other DNA
ggttaccttgttacgactt

SEQ ID NO: 3 - DNA, 41 bp, synthetic construct
  source 1..41: mol_type = other DNA
gaagtcatcatgaccgttctgcaygcnggnggnaarttyga

SEQ ID NO: 4 - DNA, 44 bp, synthetic construct
  source 1..44: mol_type = other DNA
agcagggtacggatgtgcgagccrtcnacrtcngcrtcngtcat

SEQ ID NO: 5 - DNA, 509 bp, Bacillus megaterium
  source 1..509: mol_type = other DNA
aaaggcaaaatagcttggaaatgacggcttcttccctgcttagctgaacccccggcagagtcaccctctactacatagagttcgctaatagaaggatcttttgatgaacaatctgctaatttacccggtaagtttgagatttcaagcgcgcttttacgtcttgtaagctcacgagcttttttagctgccattcttgctcttgcagccattaaacctttttcaattacctttttcgccacaatagggttctctagcaagtacgtttctaagtgttctgcaaacacagagtcagtaattgttcttgcttcactatttcccagctttgtttttgtttgtccttcgaactgcggatctgggtgcttaatagagatgatagctgtaattccttcacgaacatcttcacccgttagattggcgtcactgtctttaaatacgctgtttttacgtgcatagtcgttaattacacgcgttaacgctgttttaaatcctgcttcgtgtgttccaccttc

SEQ ID NO: 6 - DNA, 601 bp, Bacillus megaterium
  source 1..601: mol_type = other DNA
aatgattgtacgaatttcattattagataaaattttatctaaacgcgctttctctacgttgataattttaccacgtaaaggcaaaatagcttggaaatgacggcttcttccctgcttagctgaacccccggcagagtcaccctctactacatagagttcgctaatagaaggatcttttgatgaacaatctgctaatttacccggtaagtttgagatttcaagcgcgcttttacgtcttgtaagctcacgagcttttttagctgccattcttgctcttgcagccattaaacctttttcaattacctttttcgccacaatagggttctctaacaagtacgtttctaagtgttctgcaaacacagagtcagtaattgttcttgcttcactatttcccagctttgtttttgtttgtccttcgaactgcggatctgggtgcttaatagagatgatagctgtaattccttcacgaacatcttcacccgttagattggcgtcactgtctttaaatacgctgtttttacgtgcatagtcgttaattacacgcgttaacgctgttttaaatcctgcttcgtgtgttccaccttcatatgtgtgaatattg

(19) United States
(12) Patent Application Publication
(10) Pub. No.: US 2026/0033496 A1
(43) Pub. Date: Feb. 5, 2026

(54) PRIESTIA MEGATERIUM STRAINS AND METABOLITES, COMPOSITIONS COMPRISING SUCH, AND USES THEREOF

(71) Applicant: The United States of America, as represented by the Secretary of Agriculture, Washington, DC (US)

(72) Inventors: Eric T Johnson, Dunlap, IL (US); Patrick F Dowd, Peoria, IL (US); Jose L Ramirez, Peoria, IL (US)

(21) Appl. No.: 19/276,137

(22) Filed: Jul. 22, 2025

Related U.S. Application Data
(60) Provisional application No. 63/677,632, filed on Jul. 31, 2024.

(51) Int. Cl. A01N 63/20; A01N 25/00; A01P 1/00; A01P 3/00; A01P 21/00; C12N 1/20; C12R 1/01

(52) U.S. Cl. CPC A01N 63/20; A01N 25/00; A01P 1/00; A01P 3/00; A01P 21/00; C12N 1/205; C12R 2001/01

(57) ABSTRACT

Disclosed are Priestia megaterium (Pm) strains, cultures, supernatants, filtrates, extracts, and/or volatiles that are or are derived from P. megaterium strain PM10 having deposit accession number NRRL B-68352, strain PM11 having deposit accession number NRRL B-68353, or a mixture thereof. Also disclosed are methods for using such Pm strain, culture, supernatant, filtrate, extract, and/or volatile to enhance plant growth, and/or increase crop yield, and/or prevent or control pest growth and/or activity. Bioactive compositions comprising such Pm strain, culture, supernatant, filtrate, extract, and/or volatile may optionally comprise a carrier, diluent, and/or adjuvant.

### CROSS-REFERENCES TO RELATED APPLICATIONS

This application claims priority benefit from U.S. Provisional Patent Application No. 63/677,632 filed on Jul. 31, 2024. The contents of this patent application are hereby expressly incorporated by reference in their entirety.

### TECHNICAL FIELD

The disclosure is in the field of bioactive compositions comprising at least one Priestia megaterium strain and/or its metabolites, and use of such compositions in plant growth enhancement, crop yield increase, and/or prevention and control of pathogens.

### SEQUENCE LISTING

The instant application contains a Sequence Listing XML required by 37 C.F.R. § 1.831(a) which has been submitted in XML file format via the USPTO patent electronic filing system, and is hereby incorporated by reference in its entirety. The XML file was created on Jul. 29, 2024, is named 0006_24_Sequence_Listing, and has 8,000 bytes.

### BACKGROUND OF THE INVENTION

Corn is one of the most important grain crops in the world, but production is limited by a variety of pathogens and diseases. One devastating disease is caused by Phyllachora maydis and is commonly referred to as tar spot because of its characteristic black, shiny, raised leaf spots which generally range from 2-4 mm in diameter. Infection by this pathogenic fungus can result in significant yield losses, and even death of plants if infection occurs early in a susceptible variety. Tar spot disease has been endemic in much of Central and South America for several decades. It was first reported in two US “corn belt” states, Illinois and Indiana, in 2015, and since then there have been major outbreaks in several regions in both 2018 and 2021. The Crop Protection Network estimates corn growers in the US lost around $3 billion to tar spot from 2018 through 2022. Of that amount, $1.25 billion was lost in 2021 alone.

Management strategies for tar spot disease include use of resistant varieties and application of fungicides. However, under high inoculum pressure, significant yield losses can still occur, even with the use of resistant varieties. Fungicide application has not been very effective because once symptoms are noticed, further infection becomes difficult to control. At the moment there are no known corn hybrids with complete resistance to tar spot. The two common practices for reducing disease inoculum include crop rotation and tillage. However, these practices can produce mixed results and are not the sole solution for preventing tar spot.

Tar spot can move limited ranges by wind and plant residue. It overwinters in soil and residue and thrives in cool (60-70 degrees Fahrenheit) and humid conditions with prolonged periods of wet leaves. Infection can occur at any crop stage, although it's most common throughout the grain fill period. The black structures characteristic of tar spot disease are long term survival structures called stromata, which are similar to the sclerotia produced by the vegetable pathogen Sclerotinia sclerotiorum and other fungi. These stromata are the overwintering structures for tar spot, and the source of initial inoculum. However, reports of spore production and percent germination by overwintered stromata vary considerably, suggesting the presence of natural biocontrol organisms. Mycoparasites may contribute to reduced survival of stromata, as reported for other stromata or sclerotia-like structures in several fungal species.

Synthetic pesticides are often used by farmers to reduce the incidence and crop damage caused by fungi. Frequent use or misuse of synthetic pesticides has caused some countries to develop alternative products that are safer for humans and the environment. Microbial biopesticides can produce antibiotics or low molecular weight metabolites that kill or slow the growth of pathogenic fungi or bacteria. In addition, treatment of crop plants with a microbial biopesticide can induce systemic resistance in the plant that makes it more resistant to fungal pathogens.

Thus, there exists a need for new and effective microbial bioactive compositions and methods for using such in enhancing plant growth, increasing crop yield, and/or preventing, controlling, and/or reducing pathogens.

### SUMMARY OF THE INVENTION

Provided herein are Priestia megaterium (Pm) strains, cultures, supernatants, filtrates, extracts, and/or volatiles that may be used to enhance plant growth, and/or increase crop yield, and/or prevent, control, and/or reduce pest growth and/or activity.

In an embodiment, the disclosure relates to a Priestia megaterium (Pm) strain, culture, supernatant, filtrate, extract, and/or volatile that is or is derived from strain PM10 having deposit accession number NRRL B-68352, strain PM11 having deposit accession number NRRL B-68353, or a mixture thereof. In some embodiments of the disclosure, the Pm strain, culture, supernatant, filtrate, extract, and/or volatile is a plant propagation material, an inoculant composition, and/or is comprised in a bioactive composition. In some embodiments of the disclosure, the bioactive composition, Pm strain, culture, supernatant, filtrate, extract, and/or volatile is coated on to at least a portion of the outer surface of a plant, plant part, or seed of a plant. In some embodiments of the disclosure, the Pm strain, culture, supernatant, filtrate, extract, and/or volatile enhances plant growth, and/or increases crop yield, and/or prevents or controls pest growth and/or activity. In some embodiments of the disclosure, the Pm strain, culture, supernatant, filtrate, extract, and/or volatile controls growth and/or activity of unwanted fungi, nematode, microbe, insect, spider, mite, bird, fish, rodent, or deer. In some embodiments of the disclosure, the Pm strain, culture, supernatant, filtrate, extract, and/or volatile controls growth and activity of plant pathogens.

In an embodiment, the disclosure relates to a method for inhibiting the growth and/or activity of a pest in a subject or object in need thereof, the method comprising administering to the subject or object an effective amount of the at least one Pm strain, culture, supernatant, filtrate, extract, and/or volatile, or a bioactive composition comprising such, wherein the at least one Pm strain, culture, supernatant, filtrate, extract, and/or volatile is or is derived from strain PM10 having deposit accession number NRRL B-68352, strain PM11 having deposit accession number NRRL B-68353, or a mixture thereof, and wherein the bioactive composition optionally comprises a carrier and/or adjuvant. In some embodiments of the disclosure, the method inhibits growth and/or activity of unwanted fungi, nematode, microbe, insect, spider, mite, bird, fish, rodent, or deer.

In an embodiment, the disclosure relates to a method for enhancing plant growth and/or increasing crop yield, the method comprising contacting at least a portion of a plant, plant part, seed of a plant, or growth medium adjacent to a plant with a bioactive composition comprising at least one Pm strain, culture, supernatant, filtrate, extract, and/or volatile that is or is derived from strain PM10 having deposit accession number NRRL B-68352, strain PM11 having deposit accession number NRRL B-68353, or a mixture thereof.

### BRIEF DESCRIPTION OF THE DRAWINGS

These and other features, aspects, and advantages of the present disclosure will become better understood with regard to the following description, appended claims, and accompanying drawings.

FIG. 1 depicts a graph of the tar spot disease incidence Y Axis shows the percent tar spot disease, the X axis shows the year. First and fourth bars represent control; second and fifth bars represent newly identified bacterium PM10, and third and sixth bars represent newly identified bacterium PM11.

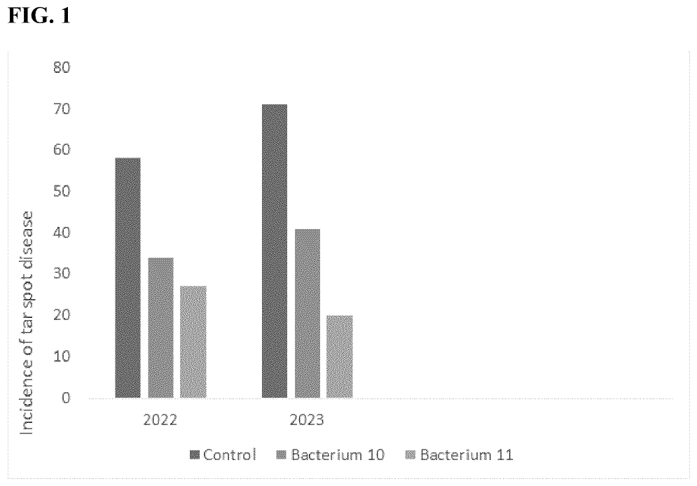

FIG. 2 depicts images of the effect of PM 10, PM 11, and Bacillus cereus on blood. Top petri dishes treated with B. cereus; center petri dishes treated with PM 10; bottom petri dishes treated with PM 11.

FIG. 3A and FIG. 3B depict images of the B37 maize leaves inoculated with F. graminearum alone or with PM10 or PM11. FIG. 3A shows a leaf inoculated with F. graminearum on the left, and with PM10 on the right. FIG. 3B shows a leaf inoculated with F. graminearum on the left, and with PM11 on the right.

### BRIEF DESCRIPTION OF THE SEQUENCE LISTING

The nucleotide sequences disclosed in the specification are listed in Table 1, below.

| Identi- | Descrip- | |
|---|---|---|
| fier | tion | Sequence |
| SEQ ID | 27F | 5′-AGAGTTTGATCCTGGCTCAG-3′ |
| NO: 1 | | |
| SEQ ID | 1492R | 5′-GGTTACCTTGTTACGACTT-3′ |
| NO: 2 | | |
| SEQ ID | UP1S | 5′-GAAGTCATCATGACCGTTCTGCA |
| NO: 3 | | YGCNGGNGGNAARTTYGA-3′ |
| SEQ ID | UP2SR | 5′-AGCAGGGTACGGATGTGCGAGCC |
| NO: 4 | | RTCNACRTCNGCRTCNGTCAT-3′ |
| SEQ ID | Pm10 | 5′-AAAGGCAAAATAGCTTGGAAATG |
| NO: 5 | gyrB | ACGGCTTCTTCCCTGCTTAGCTGAAC |
| | | CCCCGGCAGAGTCACCCTCTACTACA |
| | | TAGAGTTCGCTAATAGAAGGATCTTT |
| | | TGATGAACAATCTGCTAATTTACCCG |
| | | GTAAGTTTGAGATTTCAAGCGCGCTT |
| | | TTACGTCTTGTAAGCTCACGAGCTTT |
| | | TTTAGCTGCCATTCTTGCTCTTGCAG |
| | | CCATTAAACCTTTTTCAATTACCTTT |
| | | TTCGCCACAATAGGGTTCTCTAGCAA |
| | | GTACGTTTCTAAGTGTTCTGCAAACA |
| | | CAGAGTCAGTAATTGTTCTTGCTTCA |
| | | CTATTTCCCAGCTTTGTTTTTGTTTG |
| | | TCCTTCGAACTGCGGATCTGGGTGCT |
| | | TAATAGAGATGATAGCTGTAATTCCT |
| | | TCACGAACATCTTCACCCGTTAGATT |
| | | GGCGTCACTGTCTTTAAATACGCTGT |
| | | TTTTACGTGCATAGTCGTTAATTACA |
| | | CGCGTTAACGCTGTTTTAAATCCTGC |
| | | TTCGTGTGTTCCACCTTC-3′ |
| SEQ ID | Pm11 | 5′-AATGATTGTACGAATTTCATTAT |
| NO: 6 | gyrB | TAGATAAAATTTTATCTAAACGCGCT |
| | | TTCTCTACGTTGATAATTTTACCACG |
| | | TAAAGGCAAAATAGCTTGGAAATGAC |
| | | GGCTTCTTCCCTGCTTAGCTGAACCC |
| | | CCGGCAGAGTCACCCTCTACTACATA |
| | | GAGTTCGCTAATAGAAGGATCTTTTG |
| | | ATGAACAATCTGCTAATTTACCCGGT |
| | | AAGTTTGAGATTTCAAGCGCGCTTTT |
| | | ACGTCTTGTAAGCTCACGAGCTTTTT |
| | | TAGCTGCCATTCTTGCTCTTGCAGCC |
| | | ATTAAACCTTTTTCAATTACCTTTTT |
| | | CGCCACAATAGGGTTCTCTAACAAGT |
| | | ACGTTTCTAAGTGTTCTGCAAACACA |
| | | GAGTCAGTAATTGTTCTTGCTTCACT |
| | | ATTTCCCAGCTTTGTTTTTGTTTGTC |
| | | CTTCGAACTGCGGATCTGGGTGCTTA |
| | | ATAGAGATGATAGCTGTAATTCCTTC |
| | | ACGAACATCTTCACCCGTTAGATTGG |
| | | CGTCACTGTCTTTAAATACGCTGTTT |
| | | TTACGTGCATAGTCGTTAATTACACG |
| | | CGTTAACGCTGTTTTAAATCCTGCTT |
| | | CGTGTGTTCCACCTTCATATGTGTGA |
| | | ATATTG-3′ |

### Deposit Under Terms of Budapest Treaty

Samples of the Priestia megaterium strains identified herein have been deposited with the Agricultural Research Service Patent Culture Collection (NRRL). NRRL is located at 1815 N. University Street, Peoria, Illinois, USA 61604. All restrictions on the availability to the public of the deposited biological material identified herein will be irrevocably removed upon the granting of a patent.

Upon deposit on May 14, 2024 PM10 received NRRL Accession No. B-68352 and PM11 received NRRL Accession No. B-68353. The biological materials identified herein have been deposited under conditions such that access to the microorganisms are available during the pendency of the patent application to one determined by the Commissioner to be entitled thereto under 37 C.F.R. § 1.14 and 35 U.S.C § 122.

The deposited biological material will be maintained with all the care necessary to keep it viable and uncontaminated for a period of at least five years after the most recent request for the furnishing of a sample of the deposited microorganism, and in any case, for a period of at least thirty (30) years after the date of deposit for the enforceable life of the patent, whichever period is longer.

### DETAILED DESCRIPTION

The disclosure provides Priestia (Bacillus) megaterium (Pm) strains, cultures, supernatants, filtrates, extracts, and/or volatiles that are useful to enhance plant growth, and/or increase crop yield, and/or prevent or control pest growth and/or activity.

Tar spot of corn has caused billions of dollars in yield loss in several locations in the US over the past several years. Tar spot begins to spread from overwintered material, then spreads through a field as more stromata are formed and as they release spores. Currently there are no effective tools for controlling of tar spot, and its impact tends to be worse in irrigated fields. Based on representative labels examined, fungicides labeled for tar spot disease management state they will prevent spore germination, mycelial growth, and spore production, but do not specify whether they are active against the tar spot stromata. Stromata collected from fields and sprayed with a representative fungicide labeled for tar spot disease management still germinated (produced spores) at a high frequency. Reports indicate there is great variability in spore production by overwintered tar spot stromata, which are the source of initial inoculum to corn fields, suggesting biological agents may be killing the tar spot organism.

Described herein are microorganisms isolated from overwintered tar spot stromata collected from a field plot at the National Center for Agricultural Utilization Research (NCAUR) in Peoria, Illinois. These microorganisms have been identified as Priestia (Bacillus) megaterium (Pm), for which there have been several reports of its use in biocontrol of other fungi (S. V. Kildea, et al., 2008, “Bacillus megaterium shows potential for the biocontrol of Septoria tritici blotch of wheat,” Biol. Control 47:37-45; M. Mannaa and K. D. Kim, 2018, “Biocontrol activity of volatile-producing Bacillus megaterium and Pseudomonas protegens against Aspergillus and Penicillium spp. predominant in stored rice grains: study II,” Mycobiology 46:52-63; A. E. Saleh et al., 2021, Biocontrol activity of Bacillus megaterium BM344-1 against toxigenic fungi,” ACS omega 6:10984-10990).

Steven Kildea screened a collection of bacteria originating from barley leaves and grain, oat chaff, and wheat rhizospheres and leaves for their ability to control Septoria tritici blotch (STB) of wheat. In these trials only Bacillus megaterium (strain MKB135)] consistently retarded STB development (by up to 80%). Additional in vitro seedling studies showed that both B. megaterium cell wall components were capable of inhibiting disease development by 62%, and its culture filtrate was capable of inhibiting disease development by 36% (Kildea, S., et al., 2008, “Bacillus megaterium shows potential for the biocontrol of Septoria tritici blotch of wheat,” Biol. Control 47:37-45). P. megaterium is a rod-like, Gram-positive, mainly aerobic, spore-forming bacterium found in widely diverse habitats.

The P. megaterium isolated from overwintered tar spot stromata were deposited with the Agricultural Research Service Patent Culture Collection (NRRL). Strain PM10 received NRRL Accession No. B-68352 and strain PM11 received NRRL Accession No. B-68353.

As seen in the Examples, when tested in Petri plate assays the P. megaterium strains described herein, having NRRL Accession No. B-68352 and B-68353, inhibit growth of several species of bacteria, including Escherichia coli, Brevibacillus reuszeri, Clavibacter michiganensis ssp. michiganensis, Erwinia amylovora, and Pantoea eucalyptii. These Pm strains also inhibit the growth of Fusarium graminearum, F. proliferatum, F. verticillioides, Phyllachora maydis, and Alternaria alternata. Not wishing to be bound by theory, it is believed that these effects may be at least partly due to production of volatiles by the bacterium. Production of volatiles by other strains of Pm have been previously described (M. Mannaa and K. D. Kim, Supra; A. E. Saleh et al., Supra).

P. megaterium has been reported to control Septoria tritici blotch (Kildea S. et al. Supra) of wheat caused by ascomycete fungus, Mycophaerella graminicola, multiple species of mycotoxigenic fungi (A. E. Saleh et al. Supra), and other fungal pathogens in diverse crops (Walterson A. M. et al., 2015, “Pantoea: Insights into a highly versatile and diverse genus within the Enterobacteriaceae,” FEMS Microbiol. Rev. 39:968-974). Other Priestia species have also been reported as biocontrol agents (Bashir S. et al., 2021, “Screening of sunflower associated bacteria as biocontrol agents for plant growth promotion,” Arch. Microbiol. 203:4901-4912).

The potential for the P. megaterium strains described herein, having NRRL Accession No. B-68352 and B-68353 to secrete toxins that may cause haemolytic activity against sheep erythrocytes was tested. As seen in Example 3, the P. megaterium strains PM10 and PM11 do not secrete toxins that cause haemolytic activity against sheep erythrocytes.

The ability of the two P. megaterium strains identified herein (PM10 and PM11) to enhance corn seedling resistance to Fusarium graminearum leaf infection was tested, and the results shown in Example 4. The data showed that application of PM10 or PM11 bacteria to germinated N6 corn seeds reduced F. graminearum disease symptoms in leaves of the young plants.

The ability of the two P. megaterium strains identified herein (PM10 and PM11) to secrete volatiles that may affect fungal growth was tested. As seen in Example 5, PM10 and PM11 produced volatiles that reduced the spread of fungal colonies.

The pathogenicity of the two P. megaterium strains identified herein (PM10 and PM11) to maize was tested. As seen in Example 6, the two strains were nonpathogenic to maize when tested on maize leaves.

Different formulations can be prepared with the Pm described herein depending on the desired application method. The P. megaterium strains reported herein can be applied to the soil, to the seed, or as a foliar application in a variety of forms including liquids and solids of various formulations, such as those described herein.

The Pm described herein may be obtained by growing cells of the respective bacterial strain in liquid monoculture using well-known bacterial culture techniques. The cells are grown to high density to induce sporulation. The spores can be applied as an aqueous suspension obtained directly from the fermentation process described above, or, if the spores are purified or concentrated using methods such as ultra-filtration, centrifugation, spray-drying or freeze-drying, they should be re-suspended in water before application to crops. When the spores are applied as an aqueous suspension taken directly from the fermentation broth, other substances present in the broth will also be applied to the crops. These non-viable substances, such as bacterial metabolites or un-utilized microbial nutrients, will be applied to the plants in very small concentrations. This level of non-viable substance will not deleteriously affect the crop.

The Pm strains described herein may be applied to any type of grain, and to both conventional and hybrid varieties. During grow-out, applications of the spore suspension can be made manually, by backpack sprayer or by a more sophisticated mode such as by helicopter spraying or by any mechanical spraying device known for use in farming practice.

Spores from the Pm strains can be applied to crops by direct application to the soil, coating of the seeds prior to planting, spraying on the soil, spraying on crops after the seeds germinate, or within two weeks of the seedlings emerging. The bioactive composition may be applied to the soil, to the plant foliage, to the plant seeds, during sowing of said plant seeds, or after said plants germinate. The bioactive composition may be applied after a period of rain or watering of said plants. The bioactive composition may be applied within 10 days of sowing of the plant seeds, optionally within 3, 5, or 7 days of sowing the seeds. The bioactive composition may be applied before germination, optionally within 1, 2, 3, 4, 5, 6, 7, 8, 9, or 10 days of sowing the seeds. The bioactive composition may be applied after germination, optionally 1, 2, 3, 4, 5, 6, 7, 8, 9, or 10 days after germination.

A bioactive composition comprising at least one Pm strain, culture, supernatant, filtrate, extract, and/or volatile that is or is derived from strain PM10 having deposit accession number NRRL B-68352, strain PM11 having deposit accession number NRRL B-68353, or a mixture thereof, may be applied by spraying plants or mixing into soil. The bioactive composition may be applied to the root zone. The bioactive composition may be applied around the seed of the plant. The bioactive composition may be admixed with a soil, and the bioactive composition/soil mixture may be applied to plants, seeds, or seedlings. The bioactive composition may be applied at any temperature appropriate for field work, because if the temperature is not suitable for germination, then the spores will lie dormant until an adequate temperature occurs. The composition may be applied within 2 weeks of plant emergence. The plants may be dipped into a liquid bioactive composition. The plants may be dipped for about 1-30 seconds or 30 seconds and then planted. The plants may be treated a second time, by spraying the plants about 14 days after treatment by dipping. These microorganisms may be useful for growing season control as well, or for use against other pathogens.

Growers prefer to use pathogen control agents applied to seed as an additional application step is not needed, given that the material is applied when planting. Seed coating with microbial agents that defend against pathogens is a common technology and there are currently several products on the market, which have the material already applied to the seed, or which allow for application to seed prior to or during planting.

As used herein, “carrier” refers to an inert, organic or inorganic material, with which the active material is mixed or formulated to facilitate its application to a plant, plant part, or other object or subject to be treated, or for its storage, transport, and/or application. An agriculturally-acceptable carrier may be at least one of a buffering agent, a wetting agent, a coating agent, an abrading agent, or a mixture thereof. A carrier may be a solid particle, such as clay to which a biocontrol composition is adhered, or may be a liquid,

A biocontrol composition comprising at least one Pm strain, culture, supernatant, filtrate, extract, and/or volatile, wherein the at least one Pm strain, culture, supernatant, filtrate, extract, and/or volatile is or is derived from strain PM10 having accession No. NRRL B-68352, or strain PM11 having accession No. B-68353, or a mixture thereof, may optionally comprise at least one other biological or chemical compound or agent. A biological or chemical compound or agent may comprise a fertilizer, an insecticide, a compatible fungicide, a nematocide, or a mixture thereof. A biocontrol composition comprising at least one Pm strain, culture, supernatant, filtrate, extract, and/or volatile as taught herein, may further comprise at least one buffering agent, wetting agent, coating agent, abrading agent, clay, polysaccharide, or a mixtures thereof. A biocontrol composition comprising at least one Pm strain, culture, supernatant, filtrate, extract, and/or volatile as taught herein, may be combined with water, nutrient media, or other liquid to provide a liquid composition. A biocontrol composition comprising at least one Pm strain, culture, supernatant, filtrate, extract, and/or volatile as taught herein, may be combined with at least one solid, such as soil, a soil amendment, or other solids to provide a solid composition. A biocontrol composition comprising at least one Pm strain, culture, supernatant, filtrate, extract, and/or volatile as taught herein, may be encapsulated by, for example, polymeric materials including, but not limited to, polysaccharides to provide an encapsulated formulation. Methods and compositions for encapsulating bacteria, for example, alginate compositions, are well known in the art. Encapsulated formulations comprising a biocontrol composition comprising at least one Pm strain, culture, supernatant, filtrate, extract, and/or volatile as taught herein may be combined with liquids or solids and provided to plants by contacting plants or plant parts, or seeds or plants grown from such seeds by contacting the plants, seeds or plants grown from contacted seeds, or by contacting the soil, media, liquid or structures contacting the plants, plant parts, seeds and/or plants grown from contacted seeds. A biocontrol composition may be a liquid, solid, a semi-solid, or a mixture, admixture, suspension, or solution thereof.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

As used herein, the term “derived from” refers to a substance or organism directly isolated or obtained from a particular source, or alternatively having identifying characteristics of a substance or organism isolated or obtained from a particular source. In the event that the source is an organism, “derived from” means that it may be isolated or obtained from the organism itself or from the medium used to culture and grow said organism.

As used herein, the term “effective amount” is meant the amount of bioactive composition required to treat or prevent an infection or disease associated with a plant fungal or bacterial pathogen, or to enhance plant growth, and/or increase crop yield. The effective amount of a bioactive composition comprising at least one P. megaterium strain, culture, supernatant, filtrate, extract, and/or volatile that is or is derived from strain PM10 having deposit accession number NRRL B-68352, strain PM11 having deposit accession number NRRL B-68353, or a mixture thereof in the methods described herein varies depending upon the manner of administration, such as by coating at least a portion of a plant seed, a plant part, or a growth medium adjacent to a plant. Based on the teachings described herein, a person of skill in the art will decide the appropriate amount and dosage regimen to be used in each instance. Such amount is referred to as an “effective amount.”

The term “pest” is used to refer to any living thing such as a plant, animal, or a microorganism that has a negative effect on humans. The pest may be at least one of an unwanted plant, fungi, nematode, microbe, insect, spider, mite, bird, fish, rodent, or deer. Labeling an organism a pest is a subjective concept that varies with each individual's viewpoint. In general, pests are unwanted or undesirable because they reduce the availability, quality, or value of human resources; injure humans, animals, crops, structures, and/or possessions; spread or cause disease; or interfere with human activities by causing annoyance, discomfort, or inconvenience.

As used herein, the term “pathogen” relates to a bacterium, virus, fungal, or other microorganism that can cause disease.

Symbiosis is a broad term that includes parasites, pathogens, and mutualistic or beneficial interactions, although most associate symbiosis as having a positive or mutualistic relationship between the host and the symbiont. Symbionts in arthropods may be Eubacteria, fungi, yeasts, viruses, protozoa, or Archaea. Symbiosis enables organismal evolution and diversification in a variety of ways. Symbionts may be obligatory or facultative in relation to their arthropod host. Many insects harbor multiple symbionts, including obligatory and one or more facultative organisms. Symbionts may provide nutrients, affect host range, temperature tolerance, longevity, fecundity, sex ratio, behavior, responses to natural enemies, or other aspects of their biology.

As used herein, the term “agriculturally acceptable” refers to those compounds, materials, compositions, and/or dosage forms, which are suitable for contact with plants, without a detrimental response on the plant, or other problem complications commensurate with a reasonable benefit/risk ratio.

As used herein, the term “bioactive composition” refers to a mixture containing at least one Pm described herein and optionally at least one agriculturally-accepted carrier.

As used herein, the term “excipient” refers to a substance formulated alongside the active ingredient of a bioactive composition. At least one excipient may be included, for example, for the purpose of long-term stabilization, or to confer an enhancement on the active ingredient in the final dosage form.

Any carrier suitable for agricultural use can form part of the bioactive compositions of the present disclosure. The carrier may be any one or more of a number of carriers that confer a variety of properties, including increased stability, wettability, dispersability, etc. Wetting agents such as natural or synthetic surfactants, which can be nonionic or ionic surfactants, or a combination thereof can be included in a composition of the invention. Water-in-oil emulsions can also be used to formulate a composition that includes at least one isolated PM. Suitable formulations that may be prepared include wettable powders, granules, gels, agar strips, agar pellets, microencapsulated particles, liquids such as aqueous flowables, aqueous suspensions, water-in-oil emulsions, etc. The bioactive composition may include an agriculturally-acceptable carrier.

The agriculturally-acceptable carrier may be soil or plant growth medium, water, plant-based oils, humectants, or a combination thereof. The agriculturally-acceptable carrier may be a solid, such as diatomaceous earth, loam, silica, alginate, clay, bentonite, vermiculite, a seed case, other plant and/or animal product or combination, including granules, pellets, or suspensions. Mixtures of any of the aforementioned ingredients are also contemplated as carriers, such as but not limited to, pesta (flour and kaolin clay), agar or flour-based pellets in loam, sand, clay, etc. Formulations may include food sources for the at least one Pm strain, such as barley, rice, sugar cane bagasse, hulls or stalks from grain processing, ground plant material (“yard waste”) or wood from building site refuse, sawdust or small fibers from recycling of paper, fabric, or wood. Other suitable formulations will be known to those skilled in the art.

As used herein, the terms “metabolites” and “volatiles” are used interchangeably, and refer to a chemically diverse class of low molecular weight organic compounds having an appreciable vapor pressure under ambient conditions. Metabolites or volatiles produced by the Pm disclosed herein provide defense against pathogens.

Other than in the operating examples, or where otherwise indicated, all numbers expressing quantities of ingredients, concentrations, or reaction conditions used herein should be understood as modified in all instances by the term “about.”

As used herein, the term “about” is defined as plus or minus ten percent of a recited value. For example, about 1.0 g means 0.9 g to 1.1 g.

Mention of trade names or commercial products in this disclosure is solely for the purpose of providing specific information and does not imply recommendation or endorsement of the trade name or commercial product.

While this disclosure may be embodied in many different forms, there are described in detail herein specific preferred embodiments of the invention. The disclosed herein is an exemplification of the principles of the invention and is not intended to limit the invention to the particular embodiments illustrated. All patents, patent applications, scientific papers, and any other referenced materials mentioned herein are incorporated by reference in their entirety. Furthermore, the invention encompasses any possible combination of some or all of the various embodiments and characteristics described herein and/or incorporated herein. In addition, the invention encompasses any possible combination that also specifically excludes any one or some of the various embodiments and characteristics described herein and/or incorporated herein.

The amounts, percentages and ranges disclosed herein are not meant to be limiting, and increments between the recited amounts, percentages and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Thus, in view of the above, there is described (in part) the following:

A Priestia megaterium (Pm) strain, culture, supernatant, filtrate, extract, and/or volatile that is or is derived from strain PM10 having deposit accession number NRRL B-68352, strain PM11 having deposit accession number NRRL B-68353, or a mixture thereof.

A plant propagation material comprising at least one of the above Pm strain, culture, supernatant, filtrate, extract, and/or volatile.

A coated plant, plant part, or seed of a plant, wherein at least a portion of the outer surface of the coated plant, plant part, or seed of a plant is coated with the above Pm strain, culture, supernatant, filtrate, extract, and/or volatile.

The above Pm strain, culture, supernatant, filtrate, extract, and/or volatile, wherein the Pm strain, culture, supernatant, filtrate, extract, and/or volatile enhances plant growth, and/or increases crop yield, and/or prevents or controls pest growth and/or activity.

An inoculant composition comprising the above at least one Pm strain, culture, supernatant, filtrate, extract, and/or volatile.

A bioactive composition comprising at least one of the above Pm strain, culture, supernatant, filtrate, extract, and/or volatile; and optionally comprising a carrier; wherein the bioactive composition enhances plant growth, and/or increases crop yield, and/or prevents or controls pest growth and/or activity.

The above bioactive composition, wherein the pest is an unwanted fungi, nematode, microbe, insect, spider, mite, bird, fish, rodent, or deer.

The above bioactive composition, wherein the pest is a plant pathogen.

The above bioactive composition, wherein the pest is an arthropod symbiont.

The above bioactive composition, wherein the pest is a Phyllachora maydis, F. graminearum, F. proliferatum, Alternaria alternata, F. verticillioides, Escherichia coli, Brevibacillus reuszeri, Clavibacter michiganensis ssp. michiganensis, Erwinia amylovora, Pantoea eucalyptii, Xanthomonas campestris, Bipolaris maydis, Colletotrichum graminicola, Exserohilum turcicum, Pythium sylvaticum, or mixtures thereof.

A method for inhibiting the growth and/or activity of a pest in a subject or object in need thereof, the method comprising administering to the subject or object an effective amount of a bioactive composition comprising at least one isolated Pm strain, culture, supernatant, filtrate, extract, and/or volatile, wherein the at least one Pm strain, culture, supernatant, filtrate, extract, and/or volatile is or is derived from strain PM10 having deposit accession number NRRL B-68352, strain PM11 having deposit accession number NRRL B-68353, or a mixture thereof, and wherein the bioactive composition optionally comprises a carrier and/or adjuvant.

The above method, wherein the pest is an unwanted fungi, nematode, microbe, insect, spider, mite, bird, fish, rodent, or deer.

The above method, wherein the pest is a plant pathogen.

The above method, wherein the pest is a Phyllachora maydis, F. graminearum, F. proliferatum, Alternaria alternata, F. verticillioides, Escherichia coli, Brevibacillus reuszeri, Clavibacter michiganensis ssp. michiganensis, Erwinia amylovora, Pantoea eucalyptii, Xanthomonas campestris, Bipolaris maydis, Colletotrichum graminicola, Exserohilum turcicum, Pythium sylvaticum, or mixtures thereof.

The above method, wherein the subject is an insect, a nematode, a plant, a plant part, a seed of a plant, or a growth medium adjacent to a plant.

The above method, wherein the bioactive composition is applied to the growth medium adjacent to the plant, to at least a portion of the plant seed, the plant part, or the plant.

The above method, wherein the bioactive composition is coated onto at least a portion of the outer surface of the insect, the nematode, the plant seed, the plant part, or the plant.

A method for enhancing plant growth and/or increasing crop yield, the method comprising contacting at least a portion of a plant, plant part, seed of a plant, or growth medium adjacent to a plant with a bioactive composition comprising at least one isolated Pm strain, culture, supernatant, filtrate, extract, and/or volatile, wherein the at least one Pm strain, culture, supernatant, filtrate, extract, and/or volatile is or is derived from strain PM10 having deposit accession number NRRL B-68352, strain PM11 having deposit accession number NRRL B-68353, or a mixture thereof.

The above method, wherein the method comprises introducing a coated plant seed into a plant growth medium, wherein at least a portion of the outer surface of the plant seed is coated with the bioactive composition.

Embodiments of the present disclosure are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

### EXAMPLES

Having now generally described this invention, the same will be better understood by reference to certain specific examples, which are included herein only to further illustrate the invention and are not intended to limit the scope of the invention as defined by the claims.

### Example 1

### Isolation and Identification of Organisms

Two Priestia (Bacillus) megatarium (Pm) isolated from dead (or non-germinating stromata) were identified in stromata from overwintered corn leaves collected in Illinois, USA.

Several overwintered corn leaves of inbred GE440 (planted from seed increased from plants originally obtained from the USDA-ARS Plant Introduction Center) were collected in late April 2022 from a 2021-planted research plot. This site in Peoria, IL, has had continuous corn production for several years and is thus more ecologically stable and likely to yield biocontrol agents for tar spot than commercial fields where crop rotation typically occurs. Stromata were cut from leaves along with a small leaf piece “handle”. Approximately 50 stromata were surface-sterilized with 70% ethanol and then blotted dry as described by C. L. Groves et al. (2020, “Phyllachora maydis ascospore release and germination from overwintered corn residue,” Plant Health Prog. 21:26-30). Briefly, stromata were placed in Petri dishes with tight fitting lids (Falcon® 351006, Corning Inc.; Corning, New York, USA) containing 5 mL of 3% water agar to induce rehydration and stimulate growth of mycoparasites; in some cases, a few μL of sterile water was added to help with rehydration. Plates were held at 25-27° C. Stromata were examined daily for outgrowth of organisms that were not visually the same as any seen from the attached leaf material, and organisms noted were photographed with cameras equipped with macro lenses. Microorganisms were isolated using two different methods.

In the first method, fungal mycelium growing out of a single stroma was transferred to a potato dextrose agar (PDA, Difco potato dextrose broth, Becton Dickinson Company; Sparks, Maryland, USA, with bacto agar at 20 g/L) plate using a sterile metal probe. A few days later, bacteria were observed growing together with the fungus. The bacteria were transferred to a new PDA plate, whereas fungus in plates contaminated with bacteria was transferred to a PDA plus 0.01% chloramphenicol plate. In the second method, petroleum jelly was used to stick leaves with stromata to the inside of the top lid over a 3% water agar plate. After 2-3 days, microorganisms growing on the surface of the agar were transferred to nutrient media plates (Luria broth (LB) or tryptone glucose yeast extract (TGY) for bacteria, or PDA for fungi). LB agar plates consisted of tryptone (10 g/L), sodium chloride (10 g/L), yeast extract (5 g/L), and bacto agar at 15 g/L. TGY agar plates consisted of tryptone (5 g/L), yeast extract (5 g/L), K2HPO4 (1 g/L), and glucose (1 g/L); the pH of the final mixture was adjusted to 7.0 and bacto agar was added at 15 g/L before autoclaving.

Genomic DNA was isolated from bacteria as described by E. T. Johnson, et al., (Lett. 2014, “Expression of a wolf spider toxin in tobacco inhibits the growth of microbes and insects,” Biotechnol. 36:1735-1742 with several modifications. A 1 mL aliquot of bacterial overnight culture was centrifuged at 16,000×g; the bacterial pellet was first suspended in 480 μL of 50 mM EDTA and 120 μL of 5 mg/mL lysozyme was then added. The suspended pellet was incubated at 37° C. for 30 to 60 minutes, and the suspension was centrifuged at 16,000×g for 2 minutes. The supernatant was moved to a new tube and 600 μL of nuclei lysis solution was added. The lysate was incubated at 80° C. for 5 minutes. After cooling to room temperature, 12 μg of ribonuclease A was added to the lysate and the mixture was incubated at 37° C. for 15 to 60 minutes. Then, 250 μL of 5 M NaCl was added to the lysate and the mixture was vortexed for 20 seconds. The mixture was kept on ice for 5 minutes and then centrifuged at 16,000×g for 3 minutes. The method was continued as previously described (Johnson, E. T. et al., 2014, “Expression of a wolf spider toxin in tobacco inhibits the growth of microbes and insects” Biotechnol. Lett. 36:1735-1742).

Genomic DNA was stored at −20° C. until processing. PCR amplification of various gene products from bacterial genomic DNA was performed using Platinum™ SuperFi™ II DNA polymerase (Thermo Fisher Scientific; Waltham, Massachusetts, USA) according to the manufacturer's instructions.

Conserved gene sequences targeting the 16S rDNA gene were amplified by PCR using the primers 27F, 5′-AGAGTTTGATCCTGGCTCAG-3′, set forth in SEQ ID NO: 1, and 1492R, 5′-GGTTACCTTGTTACGACTT-3′, set forth in SEQ ID NO: 2. PCR products were sequenced using the BigDye™ Terminator Cycle Sequencing Kit (Version 3.1, Applied Biosystems; Foster City, California, USA) and Basic Local Alignment Search Tool (BLAST) analysis (National Center for Biotechnology Information (NCBI) was used to determine potential identities (Johnson, M. et al., 2008, “NCBI BLAST: A better web interface,” Nucleic Acids Res. 36: W5-W9). In some cases of bacterial identification, a portion of the gyrB gene was also amplified from genomic DNA because the 16S rDNA gene sequence was not suitable to identify the bacterium to the species level. The primers UPIS, 5′-GAAGTCATCATGACCGTTCTGCAYG CNGGNGGNAARTTYGA-3′, set forth in SEQ ID NO: 3, and UP2SR, 5′-AGCAGGGTACGG ATGTGCGAGCCRTCNACRTCNGCRTCNGTCAT-3′, set forth in SEQ ID NO: 4, were used to PCR amplify the gyrB gene fragment, and subsequently the DNA sequence was identified. The threshold for assigning a bacterial sequence to the species level was 98% or greater similarity of the PCR product sequence with a GenBank™ accession from the nucleotide collection or the whole genome shotgun contig database.

The partial gyrB sequence from B-68352, Priestia megaterium 10, was set forth in SEQ ID NO: 5, while the partial gyrB sequence from B-68353, Priestia megaterium 11, was set forth in SEQ ID NO: 6.

The information given in this example describes the identification of two newly recognized P. megaterium strains from overwintered corn leaves of inbred GE440, for which tar spot was noted on leaves when grown previously at NCAUR, and which have not been identified before.

### Example 2

Effect of Isolated P. megaterium on Bacteria and Fungi

The two P. megaterium (PM10 and PM11) bacteria identified in Example 1 were tested for their potential to serve as biocontrol agents.

Corn seed of inbred GE440, for which tar spot was noted on leaves when grown previously at the National Center for Agricultural Utilization Research (NCAUR), was coated with two strains of Pm (one strain on one batch of seed) using 1% (w/v) methocel (a common material used to coat seed) and planted in a field plot at NCAUR in early summer of both 2022 and 2023. A seasonally late planting was used as previous study has indicated tar spot is more likely to be present when corn is planted later than normal. At least 25 kernels per row were planted with at least nine rows of each treatment: seed coated with coating material alone, seed coated with Pm strain 10 or seed coated with Pm strain 11. Different treatment rows were distributed throughout the plot. Plots were monitored for tar spot presence and rated when it was found to be distributed throughout the plot.

For 2022, plant germination was good and approximately 250 plants receiving each treatment were rated for presence or absence of tar spot on leaves. In 2023, germination was not as good, presumably due to heavy rains that fell just after planting, and plant heights were variable. Initial examination of plants indicated tar spot was common on the lower leaves regardless of height, so statistical analysis was performed on the frequency of plants with more than 20 total spots on the lower two leaves, and at least 46 plants of each treatment were examined.

In 2022, the frequency of any tar spot stromata on leaves from one leaf below the car to the top leaf was: Control, 57.7%, N=279; Pm 10, 33.7%, N=264; Pm 11, 26.6%, N=256. Chi square analysis indicated: control compared to Pm 10, P=<0.0001; Control compared to Pm 11, P<0.0001; Pm 10 compared to Pm 11, P=0.0758. In 2023, the frequency of the bottom two leaves of corn plants with a combined total of greater than 20 tar spots was: Control-70.8%, N=89, Pm 10-41.1%, N=56, Pm 11-19.6%, N=46. Chi square analysis indicated: control compared to Pm 10, P=0.0004; Control compared to Pm 11, P<0.0001; Pm 10 compared to Pm 11, P=0.0198.

Additional laboratory studies indicated these P. megaterium strains can inhibit growth of several species of bacteria when tested in Petri plate assays, including Escherichia coli Brevibacillus reuszeri, Clavibacter michiganensis ssp. michiganensis, Erwinia amylovora, and Pantoea eucalyptii. These Pm strains can also inhibit the growth of the mycotoxigenic Fusarium fungi F. graminearum, F. proliferatum, and F. verticillioides in Petri plate assays, suggesting benefits beyond that of tar spot control. Presumably this is at least partly due to production of volatiles, which has been described in the past for other strains of Pm (Mannaa and Kim, Supra, Saleh et al., Supra).

The results obtained in this example show that P. megaterium strains PM10 and PM11 are useful in protecting seeds and/or plants by inhibiting growth of bacteria or fungi.

### Example 3

Effect of Isolated P. megaterium on Sheep Erythrocytes

The two P. megaterium (PM10 and PM11) bacteria identified in Example 1 were tested for their potential to secrete toxins that may cause haemolytic activity against sheep erythrocytes.

The possibility that the Priestia megaterium strains PM10 and PM11 secrete any toxin(s) that cause haemolytic activity against sheep erythrocytes was tested. Petri dishes containing tryptone soya agar (casein soya bean digest agar) with 5% defibrinated sheep blood were purchased from Thermo Fisher Scientific (Waltham, Massachusetts, USA). Each bacteria strain was inoculated onto the blood Petri dishes and incubated at 30° C. Bacillus cereus NRRL B-3711 (ATCC 14579), which secretes the toxin haemolysin BL was used as a positive control.

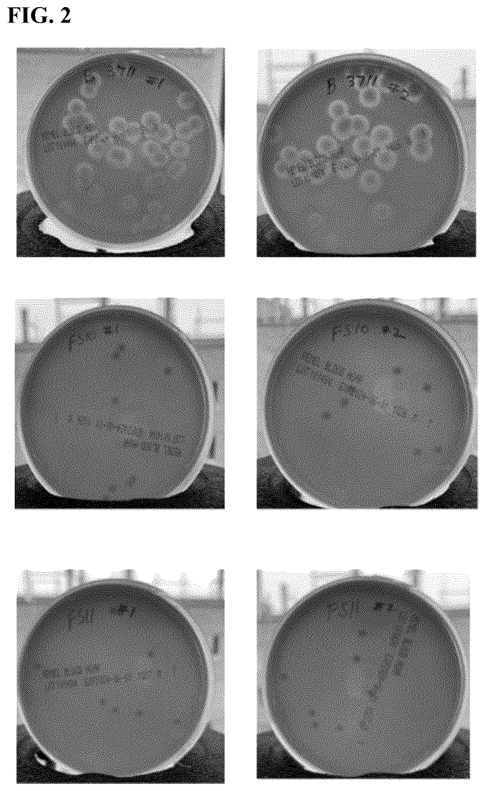

The results are shown in FIG. 2, where the positive controls are found in the top two images. These results show that there was a clearing of blood surrounding each colony in the positive controls. No clearing could be seen around the colonies of the P. megaterium strains PM10 and PM11.

The results obtained in this example show that P. megaterium strains PM10 and PM11 do not secrete toxins that cause haemolytic activity against sheep erythrocytes.

### Example 4

Effect of P. megaterium on F. graminearum Infection

The two P. megaterium (PM10 and PM11) bacteria identified in Example 1 were tested for their potential to enhance corn seed resistance to Fusarium graminearum leaf infection.

Inbred N6 corn seed was placed on wetted filter paper in Petri dishes. After the radicle had emerged in most of the seeds, the germinated seeds were soaked in a LB PM10 or PM11 culture (6700 CFUs μl⁻¹) for 10 minutes; LB medium without any microorganism served as the control. It was ensured that the radicle of each seedling was in contact with the culture. The seedlings were blotted dry and air dried in a biological safety cabinet for 25 minutes. Each seedling was placed in a small pot containing Miracle® Gro® potting soil (Miracle Gro Lawn Products, Marysville, Ohio, USA) that had been thoroughly drenched with distilled water before sowing. The plants were placed in a temperature-controlled chamber with lighting provided by fluorescent and incandescent bulbs. The mean photosynthetic photon flux density was 523±32 (SD) μEinstein m⁻² s⁻¹ when the light sensor was 66 cm from the fluorescent bulbs, based on nine measurements. The chamber was set to 16 hour of light at a temperature of 23° C. while the dark temperature was 18° C. The plants were watered with deionized water as needed. After several days the seedlings received a fertilizer treatment (Osmocote Plus tablets, 15-8-11, Scotts-Sierra, Marysville, Ohio; or Peters Professional water-soluble fertilizer, 20-20-20, JR Peters Inc., Allentown, Pennsylvania, USA). The PM10 and PM11 treated seedlings were grown until the 4 and 7 leaf stage and the second and third leaf from the top of the plant was used for the bioassay, respectively. F. graminearum spores were collected as previously described (Johnson, E T, et al., 2015, “Antifungal activity of a synthetic cationic peptide against the plant pathogens Colletotrichum graminicola and three Fusarium species,” Plant Path. J. 31:316-321) and diluted to 10,000 spores ml⁻¹ in 0.01% Triton® X-100 non-ionic surfactant (also known as polyoxyethylene octyl phenyl ether). A 2-mm slit was cut with a sharpened screwdriver in six approximately equally-spaced locations on one leaf and 2 μL of the F. graminearum spore stock was added to each slit (Dowd, P F, et al., 2012, “Enhanced pest resistance of maize leaves expressing monocot crop plant-derived ribosome-inactivating protein and agglutinin,” J. Agric. Food Chem. 60:10768-10775). The width of each leaf lesion was measured to the nearest mm and noted daily for up to 3 days (Dowd et al., Supra). Significant differences in F. graminearum lesion size were determined by analysis of variance using SAS Proc GLM version 9.4 (SAS Institute, Cary, North Carolina, USA; the SAS Proc GLM analyzes data within the framework of general linear models).

TABLE 2
F. graminearum lesion sizes

| Treatment | Size of lesion (mm) |
|---|---|
| Control | 6.3 ± 0.4 a |
| Treated with PM10 bacteria | 4.5 ± 0.4 b |
| Control | 3.1 ± 0.3 a |
| Treated with PM11 bacteria | 1.9 ± 0.3 b |

As seen above in Table 2, application of PM10 or PM11 bacteria to germinated N6 corn seeds enhanced resistance to Fusarium graminearum leaf infection in young plants. Values in the same column with different letters were statistically different by ANOVA at p<0.05.

The results obtained in this example show that application of PM10 or PM11 to germinated corn seeds enhances resistance to F. graminearum.

### Example 5

Effect of P. megaterium Volatiles on Fungal Colonies

The two P. megaterium (PM10 and PM11) bacteria identified in Example 1 were tested for their potential to secrete volatiles that may effect fungal growth.

PM10 and PM11 cultures were grown in LB medium with shaking (200-250 revolutions per minute) overnight at 30° C. Each culture was diluted 1:100 with Ringer's solution (7.2 g NaCl, 0.17 g CaCl₂), and 0.37 g KCl per L of water, pH 7.3-7.4). A hundred microliters of this dilution were added to one Petri dish (100×15 mm) containing BBL™ Trypticase™ soy broth with Difco™ agar (15 g 1-1, abbreviated as TSA; BBL™ Trypticase™) media for bacteriological laboratory cultures as trademarked by Becton Dickinson and Company. This plate served as the source of volatiles produced by the P. megaterium bacteria and contained an estimated 50,000-190,000 colony forming units (CFUs). The concentration of CFUs in the original P. megaterium culture used for each experiment was calculated by making two more 1:100 serial dilutions and spreading 100 μL of the last dilution onto one TSA Petri dish and counting the CFUs on the Petri dish after sitting overnight in an incubator set to 30° C. The mean number of CFUs was calculated from triplicate dishes of the most dilute suspension of bacteria. The amount of CFUs in each experiment is noted in Table 3 below.

TABLE 3
CFU per dish

| | Test A | | Test B | |
|---|---|---|---|---|
| Organism receiving volatiles | PM10 | PM11 | PM10 | PM11 |
| Fusarium verticillioides | 50,000 | 130,000 | 60,000 | 190,000 |
| Fusarium proliferatum | 50,000 | 130,000 | 60,000 | 140,000 |
| Alternaria alternata-or | 60,000 | 190,000 | 90,000 | 100,000 |
| arborescens* | | | | |

*Multi-locus phylogenetic analysis could not discriminate between these two related species,

Several fungal spores (the amounts for each experiment are listed in Table 4 below) were placed in the center of V8 (for Fusarium fungi) or PDA (for Alternaria fungi) medium contained in Petri dishes. The bottom part of the dish with the PM10 or PM11 bacteria was sealed to the bottom part of the V8 or PDA dish containing the fungal spores using double layers of PARAFILM moisture proof, self-sealing flat wrapper. A single layer of SCOTCH pressure sensitive adhesive tape was added on top of the PARAFILM seal, self-sealing flat wrapper, and the dishes, prepared as triplicates, were incubated at 30° C. in the dark with the PM10/PM11 dish or blank TSA dish on the bottom. The size (the largest distance from left edge to right edge possible) of the fungal mass was measured by a ruler after several days of incubation (the duration of each experiment is noted in Table 4 below).

TABLE 4
Trial Length

| | Trial A, length | Trial B, length |
|---|---|---|
| Organism receiving volatiles | in h | in h |
| Fusarium verticillioides | 96 | 96 |
| Fusarium proliferatum | 96 | 96 |
| Alternaria alternata-arborescens | 123 | 121 |

The average size of fungal colonies on a Petri dish exposed to volatiles emitted by P. megaterium strains PM10 and PM11 are listed in Table 5 below. The data in this table show that volatiles produced by PM10 and PM11 reduced the average size of Fusarium verticillioides, Fusarium proliferatum, and Alternaria alternata or arborescens.

TABLE 5
Fungal Colony Size

| | | P. megaterium PM10 | P. megaterium PM11 |
|---|---|---|---|
| Fungus | Control | volatiles | volatiles |
| Fusarium verticillioides | A 59 ± 2 (3) a | 47 ± 0.5 (2) b [80%] | 46 ± 0.6 (3) b [78%] |
| | B 45 ± 0.3 (3) a | 34 ± 0.3 (3) b [76%] | 34 ± 0.3 (3) b [76%] |
| Fusarium proliferatum | A 53 ± 0.6 (3) a | 37 ± 3 (3) b [70%] | 40 ± 0 (2) b [76%] |
| | B 40 ± 0.8 (3) a | 33 ± 0.7 (3) b [83%] | 33 ± 1 (3) b [83%] |
| Alternaria alternata or | A 41 ± 0 (3) a | 28 ± 2 (3) b [68%] | 28 ± 1 (3) b [68%] |
| arborescens* line 8C | B 39 ± 0.9 (3) a | 30 ± 0.6 (3) b [77%] | 29 ± 2 (3) b [74%] |

The results obtained in this example show that PM10 and PM11 emit volatiles that are useful for reducing the spread of fungal colonies.

### Example 6

Lack of Pathogenic Effect of P. megaterium on B37 Corn

The two P. megaterium (PM10 and PM11) bacteria identified in Example 1 were tested for their potential to act as pathogens of corn. The corn pathogen F. graminearum was used as a positive control and leaf material from corn inbred B37 was used as the test material.

Inbred corn line B37 is susceptible to many fungal pathogens, if inoculated it has some susceptibility to the bacterial disease called Stewart's wilt. This disease is caused by the bacterial pathogen Pantoea stewartii. Leaves from inbred B37 mays were inoculated with F. graminearum on one side and either PM10 or PM11 on the other, placed in Petri dishes containing 3% water agar, and the size of the lesions measured after three (3) days. Twenty colony forming units of F. graminearum and ten CFUs of PM10 and PM11 were applied.

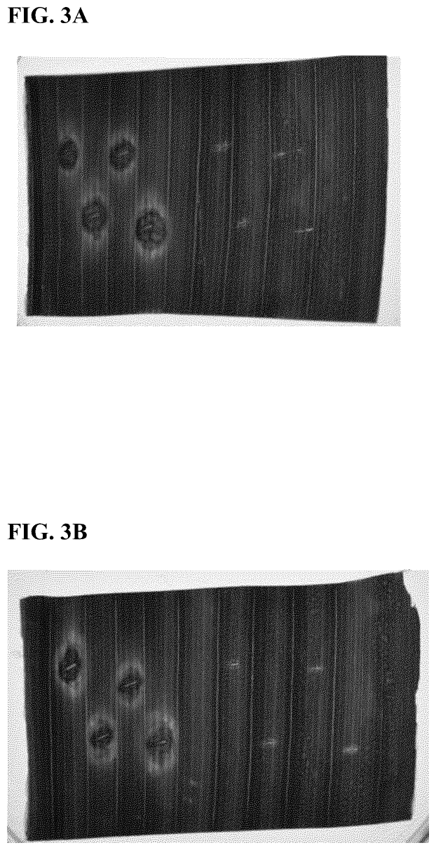

FIG. 3A shows a leaf inoculated with F. graminearum on the left, and with PM10 on the right. FIG. 3B shows a leaf inoculated with F. graminearum on the left, and with PM11 on the right. As seen on these figures, F. graminearum formed large lesions on the treated maize leaves, but neither PM10 nor PM11 formed lesions.

The data presented in this Example shows that PM10 and PM11 are not pathogenic to corn.

### Example 7

Infection-Resistance of P. megaterium-Treated Seeds

Here is some data from a recently completed experiment using seed inoculation of #11 (Eric can provide details if methodology was different than before).

A disease susceptible inbred of corn (N6) was used. Seed were coated with Pm11. Seeds were planted, and leaves from 5-leaf plants removed and used for testing.

The 3^{rd} leaf was removed from a 5-leaf plant and puncture spots were inoculated with Xanthomonas campestris, which can cause bacterial leaf streak of corn. The strain is from the ARS culture collection, accession B-65403, and was originally isolated from a tomato leaf. Six N6 inbred leaves received each treatment and there were 4 treated wounds per leaf piece, along with 4 control wounds that received inoculation with carrier only (no pathogen). Evaluations were based on mm integer width. Non-inoculated wounds had negligible lesions. On day 5, wounds on plants from control seeds were 2.2±0.2 mm, plants from Pm #11-treated seeds were 0.5±0.1 mm. By analysis of variance, F value=71.87, P<0.0001.

Seeds of maize inbred B37 were incubated at room temperature for 1 h in the following solution: Priestia megaterium 10 (Pm10) solution was diluted in Luria-Bertani (LB, Becton Dickinson, Sparks, MD, USA) medium to 9000 CFU per μl; Priestia megaterium 11 solution was diluted in LB to 47,000 CFU per μl. Then the seeds were dried in a laminar flow safety cabinet. The next day, one seed was placed in one 4-inch pot that contained pre-wetted (washed with deionized water) propagation mix soil (Sungro Horticulture, Agawam, MA, USA). The plants were grown in a large climate-controlled room with alternating 1000 W sodium and halide lighting with target temperatures of 24±2° C. in the day and 18±2° C. at night; the lights were on for 14 h each day. After 10-14 d each pot received a plug of Osmocote fertilizer (15-8-11, Everris, The Netherlands) to ensure the plants were healthy at the start of the bioassay. The second leaf from a five-leaf plant was placed in a Petri dish containing about 20 ml of water agar medium (agar at 3% v/v). Eight slits were made in each leaf using a small screwdriver. Plugs of mycelia of Bipolaris maydis, Colletotrichum graminicola, Exserohilum turcicum or Pythium sylvaticum were placed onto 1 slit, 4 slits in total, in the leaf; the other four slits received a plug of growth medium. The dish was closed tightly, and the dishes were placed in a Tupperware container that was wrapped in aluminum foil; all of the containers were placed in an incubator set for 27±1° C. Data below is based on nearest 1 mm mean+/−standard error ratings. All treated ratings were significantly different from controls at P<0.05 by analysis of variance.

TABLE

| | Control | Pm10 | Pm11 |
|---|---|---|---|
| Bipolaris maydis 2 day necrosis | 9.1 +/− 0.4 | 5.0 +/− 0.5 | 4.1 +/− 0.4 |
| Colletotrichum graminicola 4 day necrosis, non- | 3.4 +/− 0.3 | 1.9 +/− 0.1 | 2.1 +/− 0.3 |
| zero ratings only | | | |
| Exserohilum turcicum 2 day Chlorosis | 1.9 +/− 0.4 | 0.3 +/− 0.1 | 0.6 +/− 0.2 |
| Pythium sylvaticum 3 day necrosis | 1.7 +/− 0.2 | 0.8 +/− 0.2 | 0.4 +/− 0.1 |

The data in this Example shows that plants from Pm10 and Pm11-coated seeds inhibit growth of different types of pathogens

We claim:

1. A Priestia megaterium (Pm) strain, culture, supernatant, filtrate, extract, and/or volatile that is or is derived from strain PM10 having deposit accession number NRRL B-68352, strain PM11 having deposit accession number NRRL B-68353, or a mixture thereof.

2. A plant propagation material comprising at least one Pm strain, culture, supernatant, filtrate, extract, and/or volatile of claim 1.

3. A coated plant, plant part, or seed of a plant, wherein at least a portion of the outer surface of the coated plant, plant part, or seed of a plant is coated with the Pm strain, culture, supernatant, filtrate, extract, and/or volatile of claim 1.

4. The Pm strain, culture, supernatant, filtrate, extract, and/or volatile of claim 1, wherein the Pm strain, culture, supernatant, filtrate, extract, and/or volatile enhances plant growth, and/or increases crop yield, and/or prevents or controls pest growth and/or activity.

5. An inoculant composition comprising the at least one Pm strain, culture, supernatant, filtrate, extract, and/or volatile of claim 1.

6. A bioactive composition comprising at least one Pm strain, culture, supernatant, filtrate, extract, and/or volatile of claim 1; and optionally comprising a carrier;
wherein the bioactive composition enhances plant growth, and/or increases crop yield, and/or prevents or controls pest growth and/or activity.

7. The bioactive composition of claim 6, wherein the pest is an unwanted fungi, nematode, microbe, insect, spider, mite, bird, fish, rodent, or deer.

8. The bioactive composition of claim 6, wherein the pest is a plant pathogen.

9. The bioactive composition of claim 6, wherein the pest is an arthropod symbiont.

10. The bioactive composition of claim 6, wherein the pest is a Phyllachora maydis, F. graminearum, F. proliferatum, Alternaria alternata, F. verticillioides, Escherichia coli, Brevibacillus reuszeri, Clavibacter michiganensis ssp. michiganensis, Erwinia amylovora, Pantoea eucalyptii, Xanthomonas campestris, Bipolaris maydis, Colletotrichum graminicola, Exserohilum turcicum, Pythium sylvaticum, or mixtures thereof.

11. A method for inhibiting the growth and/or activity of a pest in a subject or object in need thereof, the method comprising administering to the subject or object an effective amount of a bioactive composition comprising at least one isolated Pm strain, culture, supernatant, filtrate, extract, and/or volatile, wherein the at least one Pm strain, culture, supernatant, filtrate, extract, and/or volatile is or is derived from strain PM10 having deposit accession number NRRL B-68352, strain PM11 having deposit accession number NRRL B-68353, or a mixture thereof; and wherein the bioactive composition optionally comprises a carrier and/or adjuvant.

12. The method of claim 11, wherein the pest is an unwanted fungi, nematode, microbe, insect, spider, mite, bird, fish, rodent, or deer.

13. The method of claim 11, wherein the pest is a Phyllachora maydis, F. graminearum, F. proliferatum, Alternaria alternata, F. verticillioides, Escherichia coli, Brevibacillus reuszeri, Clavibacter michiganensis ssp. michiganensis, Erwinia amylovora, Pantoea eucalyptii, Xanthomonas campestris, Bipolaris maydis, Colletotrichum graminicola, Exserohilum turcicum, Pythium sylvaticum, or mixtures thereof.

14. The method of claim 11, wherein the subject is a plant, a plant part, a seed of a plant, or a growth medium adjacent to a plant.

15. The method of claim 14, wherein the bioactive composition is applied to the growth medium adjacent to the plant, to at least a portion of the plant seed, the plant part, or the plant.

16. The method of claim 14, wherein the bioactive composition is coated onto at least a portion of the outer surface of the plant seed, the plant part, or the plant.

17. A method for enhancing plant growth and/or increasing crop yield, the method comprising contacting at least a portion of a plant, plant part, seed of a plant, or growth medium adjacent to a plant with a bioactive composition comprising at least one isolated Pm strain, culture, supernatant, filtrate, extract, and/or volatile, wherein the at least one Pm strain, culture, supernatant, filtrate, extract, and/or volatile is or is derived from strain PM10 having deposit accession number NRRL B-68352, strain PM11 having deposit accession number NRRL B-68353, or a mixture thereof.

18. The method of claim 17, wherein the method comprises introducing a coated plant seed into a plant growth medium, wherein at least a portion of the outer surface of the plant seed is coated with the bioactive composition.
